(19) United States
(12) Patent Application Publication
(10) Pub. No.: US 2025/0146032 A1
(43) Pub. Date: May 8, 2025

(54) METHOD FOR PRODUCING ¿-3 POLYUNSATURATED FATTY ACID

(71) Applicant: XIAMEN HUISON BROTECH CO., LTD, Xiamen (CN)

(72) Inventors: Liyi CHEN, Xiamen (CN); Huichang ZHONG, Xiamen (CN); Shuirong CHEN, Xiamen (CN); Meng Yin, Xiamen (CN); Shengli ZENG, Xiamen (CN)

(73) Assignee: XIAMEN HUISON BROTECH CO., LTD, Xiamen (CN)

(21) Appl. No.: 19/014,537

(22) Filed: Jan. 9, 2025

Related U.S. Application Data
(63) Continuation of application No. PCT/CN2024/090791, filed on May 30, 2024.

(30) Foreign Application Priority Data
May 4, 2023 (CN) 202310489514.7

(51) Int. Cl. C12P 7/6427

(52) U.S. Cl. CPC C12P 7/6427

(57) ABSTRACT

The present application relates to the technical field of microbial fermentation, and discloses a method for producing an ω-3 polyunsaturated fatty acid, comprising: culturing to obtain a seed liquid of Schizochytrium sp., and trans-inoculating the obtained seed liquid of Schizochytrium sp. into a fermentation medium in an inoculation amount of 30-40% for fermentation to obtain the ω-3 polyunsaturated fatty acid. The method for fermentation of Schizochytrium sp. in the present application can greatly improve the yield of various ω-3 polyunsaturated fatty acids.

### CROSS-REFERENCE TO RELATED APPLICATIONS

The present application claims the priority and benefit of China Patent Application No. 202310489514.7 filed to China National Intellectual Property Administration on May 4, 2023, the entire contents of which are incorporated herein by reference.

### TECHNICAL FIELD

The present application relates to the technical field of microbial fermentation, in particular to a method for producing an ω-3 polyunsaturated fatty acid.

### BACKGROUND

EPA, also known as eicosapentaenoic acid, is a long-chain ω-3 series of polyunsaturated fatty acids, which has many functions in regulating the physiological health of organisms, such as regulating blood pressure, blood lipid and blood sugar, anti-inflammatory and antioxidant activities, etc., and also has some potential beneficial effects on atherosclerotic plaque factors and weight loss. Since EPA cannot synthesize by itself in human body, it can only be taken from food.

DPA, also known as docosapentaenoic acid, has two structures. One is ω-3DPA, which has functions such as regulating blood lipid, softening blood vessels, reducing blood viscosity, improving vision, promoting growth and development, and enhancing immune functions of human bodies. Its function of regulating blood lipid is many times stronger than that of EPA, which is known as the vascular scavenger. It is more suitable for middle-aged and elderly people with high blood lipid, and it is also an important immune factor in human breast milk, especially an essential fatty acid in the growth of infants. The other is ω-6DPA. Studies have investigated that ω-6DPA can be reversely converted into arachidonic acid in animals, and metabolites derived from ω-6DPA can adjust the function of macrophages, relieve inflammatory diseases such as experimental colitis, and also can effectively inhibit platelet activation and thrombosis after vascular injury, thus playing a role in preventing cardiovascular diseases, and when combined with DHA, it can enhance the anti-inflammatory and neuroprotective effects of DHA. Both ω-3DPA and ω-6DPA are beneficial to improve the lipoprotein distribution of hamsters fed with a high cholesterol diet, with the former especially having a better effect on the aortic function.

DHA, also known as docosahexaenoic acid, is an indispensable ω-3 series of polyunsaturated fatty acids for human bodies, commonly known as brain gold. It has unique physiological functions in promoting brain nerve development, improving vision, relieving brain diseases, anti-inflammation, anti-cancer, enhancing immunity, enhancing lipid metabolism, maintaining intestinal and cardiovascular health, etc., and is especially crucial for the intellectual and visual development of infants.

At present, deep-sea fish oil is the main commercial source of ω-3 fatty acids, but marine microalgae are the primary producers of polyunsaturated fatty acids and have the ability of synthesizing ω-3 (such as DHA and EPA). Moreover, algae oil has no fishy smell and a very low cholesterol content. Microorganisms that can produce DHA/EPA mainly include Schizochytrium sp., Mortierella alpina, Pythium irregulare, Chlorella, Phaeodactylum tricornutum, etc. Among them, Schizochytrium sp. is an industrialized producer of DHA (docosahexaenoic acid) with a large biomass and high fatty acid content, and also one of the potential strains for industrial production of EPA and DPA.

In the prior art, there have been studies on the production of DHA, EPA or DPA from fermentation of Schizochytrium sp. However, the existing methods for fermentation of Schizochytrium sp. cannot effectively enhance the contents of DHA, EPA and DPA simultaneously. For example, a method for preparing Schizochytrium sp. oil containing EPA and DHA is disclosed in patent CN202111291970.8, which can increase the content of DHA, but the content of EPA in Schizochytrium sp. extract cannot be effectively increased.

Therefore, there is still an urgent need for a production method that can simultaneously enhance the yield of various ω-3 polyunsaturated fatty acids.

### SUMMARY

The present application provides a method for producing an ω-3 polyunsaturated fatty acid, which is used for solving the problem of low content and yield of EPA in Schizochytrium sp. extract.

The present application relates to a method for producing an ω-3 polyunsaturated fatty acid, including: culturing Schizochytrium sp. to obtain a seed liquid of Schizochytrium sp.; and trans-inoculating the obtained seed liquid of Schizochytrium sp. into a fermentation medium for fermentation, wherein pH and temperature of the fermentation are regulated in stages, a concentration of a residual sugar in the fermentation medium during the fermentation is maintained at 20-40 g/L by batch feeding, and dissolved oxygen in the fermentation process is controlled at more than 50%. 3-10 g/L of amino acids are added in a fed-batch manner at a stage of 20-48 hr after the start of the fermentation. A total fermentation time is 144-168 hrs. Optional fermentation conditions include a fermentation temperature of room temperature and a fermentation pH of 5.0-6.0. The method is beneficial to the growth of Schizochytrium sp., and to enhance the dry cell weight after fermentation as well as the content and yield of ω-3 polyunsaturate fatty acids.

The present application also relates to a method for producing an ω-3 polyunsaturated fatty acid, including:
(1) culturing Schizochytrium sp. to obtain a seed liquid of Schizochytrium sp.; and
(2) trans-inoculating the seed liquid of Schizochytrium sp. obtained in step (1) into a fermentation medium in an inoculation amount of 30-40% for fermentation at conditions as below: a fermentation temperature of 25-28° C., and a fermentation pH of 5.0-6.0, the pH and temperature of the fermentation are regulated in stages, a concentration of a residual sugar in the fermentation medium during the fermentation is maintained at 20-40 g/L by batch feeding, dissolved oxygen in the fermentation process is controlled at more than 50%, and a total fermentation time is 144-168 hrs, to obtain the ω-3 polyunsaturated fatty acid, wherein 3-10 g/L of amino acids are added in a fed-batch manner at a stage of 20-48 hrs after the start of the fermentation.

The method is beneficial to the growth of Schizochytrium sp., and to enhance the dry cell weight after fermentation as well as the content and yield of ω-3 polyunsaturate fatty acids.

The present application relates to a method for producing an ω-3 polyunsaturated fatty acid, including: culturing Schizochytrium sp. to obtain a seed liquid of Schizochytrium sp.; and trans-inoculating the obtained seed liquid of Schizochytrium sp. into a fermentation medium for fermentation, wherein pH and temperature of the fermentation are regulated in stages, a concentration of a residual sugar in the fermentation medium during the fermentation is maintained at 20-40 g/L by batch feeding, and dissolved oxygen in the fermentation process is controlled at more than 20%. Amino acids, such as a mixture of glutamic acid, lysine and/or tyrosine are added in a fed-batch manner from about the 15^{th} hr after the start of the fermentation; or amino acids are added in a fed-batch manner from about the 20^{th} hr after the start of the fermentation, the amino acids are selected from the mixture of lysine, threonine and/or arginine; or amino acids are added in a fed-batch manner from about the 15^{th} hr after the start of the fermentation, the amino acids are selected from the mixture of lysine, glycine and/or phenylalanine. In some embodiments, about 3-10 g/L of amino acids are added in a fed-batch manner at the 20-48 hr stage, or about 10 g/L of amino acids are added in a fed-batch manner at the 25-40 hr stage, or about 5 g/L of amino acids are added in a fed-batch manner at the 26-35 hr stage. In some embodiments, the total fermentation time is more than about 120 hrs, or more than about 125 hrs, or more than about 130 hrs, or more than about 135 hrs, or more than about 140 hrs, or more than about 142 hrs, or more than about 144 hrs, and the total fermentation time is preferably about 144-168 hrs.

In some embodiments, the seed liquid of Schizochytrium sp. is trans-inoculated into the fermentation medium in an inoculation amount of about 25%, about 28%, about 29%, about 30%, or about 31%, about 33%, about 35%, about 37%, about 39%, about 40%, about 41%, about 43%, about 45%, about 48%, or about 50% for fermentation. In some embodiments, the seed liquid of Schizochytrium sp. is trans-inoculated into the fermentation medium in an inoculation amount of about 30% to about 40% for fermentation.

In some embodiments, the concentration of the residual sugar in the fermentation medium during the fermentation is maintained at about 15 g/L, or about 17 g/L, or about 19 g/L, about 21 g/L, about 23 g/L, about 25 g/L, about 27 g/L, about 29 g/L, about 31 g/L, about 33 g/L, about 35 g/L, about 37 g/L, about 39 g/L, about 41 g/L, about 43 g/L, about 45 g/L, or about 47 g/L by batch feeding. In some embodiments, the concentration of the residual sugar in the fermentation medium during the fermentation is maintained at about 20 g/L to about 40 g/L by batch feeding.

In some embodiments, dissolved oxygen in the fermentation process is controlled at more than about 20%, more than about 25%, more than about 30%, more than about 35%, more than about 40%, more than about 42%, more than about 45%, more than about 47%, more than about 49%, or more than about 50%, or more than about 55%.

In some embodiments, optional fermentation conditions include a fermentation temperature of room temperature, e.g., sometimes a room temperature of about 10° C., about 15° C., about 20° C., about 25° C., about 30° C., about 35° C., or about 40° C., etc.

In some embodiments, the process of regulating the pH of the fermentation in stages includes optionally controlling the pH to be about 5.5 to about 6.0 during the 0-48 hr stage of the fermentation; and controlling the pH to be about 5.0 to about 6.0 after 48 hrs of fermentation.

In some embodiments, a method for producing an ω-3 polyunsaturated fatty acid includes:
(1) culturing Schizochytrium sp. to obtain a seed liquid of Schizochytrium sp.; and
(2) trans-inoculating the seed liquid of Schizochytrium sp. obtained in step (1) into a fermentation medium in an inoculation amount of about 30-about 40% for fermentation at conditions as below: a fermentation temperature of about 25° C. to about 28° C., and a fermentation pH of about 5.0 to about 6.0, the pH and temperature of the fermentation are regulated in stages, the concentration of a residual sugar in the fermentation medium during the fermentation is maintained at about 20 to about 40 g/L by batch feeding, dissolved oxygen in the fermentation process is controlled at more than 50%, and the total fermentation time is about 144 to about 168 hrs, to obtain the ω-3 polyunsaturated fatty acid, wherein 3-10 g/L of amino acids are added in a fed-batch manner at the 20-48 hr stage after the start of the fermentation.

In some embodiments, the amino acids are at least one of glutamic acid, lysine, tyrosine, threonine, arginine, glycine, and phenylalanine. In some embodiments, the amino acids are at least more than two of glutamic acid, lysine, tyrosine, threonine, arginine, glycine or phenylalanine. In some embodiments, the amino acids are glutamic acid, lysine, and tyrosine at a mass ratio of about 1:1:1.

In some embodiments, the process of regulating the pH of the fermentation in stages includes controlling the pH to be about 5.5-6.0 during the 0-48 hr stage of the fermentation process; and controlling the pH to be about 5.0 to about 6.0 after 48 hrs of fermentation.

In some embodiments, the pH is adjusted with an acid and/or abase in the fermentation process, and the base is a complex of one or more of ammonia water, sodium hydroxide, potassium hydroxide, sodium carbonate and calcium carbonate; and the acid is a complex of one or more of citric acid, malic acid, acetic acid, propionic acid, succinic acid, oxalic acid, phosphoric acid, sulfuric acid, hydrochloric acid and phytic acid.

In some embodiments, the process of regulating the temperature of the fermentation in stages includes controlling the fermentation temperature to be about 28° C. during the 0-48 hr stage of the fermentation process, and controlling the fermentation temperature to be about 25° C. after 48 hrs of fermentation.

In some embodiments, the fermentation medium includes: 40-60 g/L of initial glucose, 5-15 g/L of yeast extract powder, 2-5 g/L of yeast powder, 2-10 g/L of corn syrup dry powder, 10-30 g/L of anhydrous sodium sulfate, 0.1-0.2 g/L of anhydrous calcium chloride, 0.3-0.6 g/L of potassium chloride, 0.4-0.8 g/L of magnesium sulfate, 0.5-1.5 g/L of potassium dihydrogen phosphate, 0.5-1.5 g/L of ammonium sulfate, 15-40 mg/L of Vitamin B12, 5-15 mg/L of Vitamin B1, and 5-15 mg/L of biotin.

In some embodiments, in step (1), Schizochytrium sp. is cultured by a shake-flask culture, a primary seed culture and a secondary seed culture to obtain the seed liquid of Schizochytrium sp., wherein the shake-flask culture includes: inoculating Schizochytrium sp. into a shake-flask seed medium for culture to obtain a shake-flask seed liquid, wherein culture conditions of the shake-flask culture are as follows: culturing at a culture temperature of 28-30° C. and in a shake flask at 180-220 rpm for 24-30 hrs;
the primary seed culture includes: inoculating the shake-flask seed liquid into a primary seed medium in an inoculation amount of 24% to obtain a primary seed liquid; wherein culture conditions of the primary seed culture are as follows: culturing at a temperature of 28-30° C. and adjusting the pH of the fermentation to about 5.0 to about 6.0 with a base and/or an acid; and
the secondary seed culture includes: after culturing in the primary seed culture step to a residual sugar concentration in the primary seed medium of less than 20 g/L, inoculating the primary seed liquid into a secondary seed medium in an inoculation amount of 10-20% for culture to obtain the seed liquid of Schizochytrium sp.; wherein culture conditions of the secondary seed culture are as follows: culturing at a culture temperature of 28-30° C., adjusting the pH of the fermentation to 5.0-6.0 with a base and/or an acid, and culturing the secondary seed until the residual sugar in the secondary seed medium is less than 20 g/L.

In some embodiments, the shake-flask seed medium includes: 40-80 g/L of glucose, 10-15 g/L of yeast extract powder, 10-15 g/L of anhydrous sodium sulfate, 0.1-0.2 g/L of anhydrous calcium chloride, 0.3-0.6 g/L of potassium chloride, 0.5-1.5 g/L of potassium dihydrogen phosphate, 2.0-4.0 g/L of ammonium sulfate, 1.0-4.0 g/L of zinc sulfate, 2.0-6.0 g/L of magnesium sulfate, and 0.5-1.5 g/L of potassium sulfate;
the primary seed medium includes: 40-80 g/L of glucose, 5-15 g/L of yeast extract powder, 10-15 g/L of anhydrous sodium sulfate, 0.1-0.2 g/L of anhydrous calcium chloride, 0.3-0.6 g/L of potassium chloride, 0.5-1.5 g/L of potassium dihydrogen phosphate, 2.0-4.0 g/L of ammonium sulfate, 1.0-4.0 g/L of zinc sulfate, 2.0-6.0 g/L of magnesium sulfate, and 0.5-1.5 g/L of potassium sulfate; and
the secondary seed medium includes: 40-60 g/L of glucose, 5-15 g/L of yeast extract powder, 5-10 g/L of yeast powder, 10-20 g/L of anhydrous sodium sulfate, 0.1-0.2 g/L of anhydrous calcium chloride, 0.3-0.6 g/L of potassium chloride, 0.5-1.5 g/L of potassium dihydrogen phosphate, 2.0-4.0 g/L of ammonium sulfate, 1.0-4.0 g/L of zinc sulfate, 2.0-6.0 g/L of magnesium sulfate, and 0.5-1.5 g/L of potassium sulfate.

In some embodiments, the batch feeding is to add glucose as a supplement.

After fermentation for a period of time, the dry cell weight of the fermentation broth is measured, the content ratio of the crude lipid is also determined, and the content of the related ω-3 polyunsaturated fatty acid in the crude lipid is determined. In some embodiments, the dry cell weight of the fermentation broth is about 135 g/L, about 140 g/L, about 145 g/L, about 150 g/L, about 155 g/L, about 160 g/L, about 165 g/L, about 170 g/L, about 175 g/L, about 180 g/L, about 185 g/L, about 190 g/L, about 195 g/L, about 200 g/L, about 205 g/L, and about 209 g/L.

The above production method of the present application is also helpful for enhancing the content of fatty acid biomass in Schizochytrium sp., which biomass contains at least one of DHA, EPA and/or DPA, especially for enhancing the content of EPA in fermentation broth cells, wherein the mass percentage of the EPA in the dry cell weight is greater than or equal to 8.0%, or the mass percentage of the EPA in the unsaturated fatty acids in the cell lipid is greater than or equal to 15%, or about 8.0% to 15%, or about 9.0% to about 12%. In some embodiments, the content of EPA reaches more than about 8%, or more than about 8.5%, or more than about 9.0%, or more than about 9.5% or more than about 10.0%, or more than about 1²0.0% or more than about 15.0%.

A certain technical scheme above has the following beneficial effects:

In the present application, adding complex amino acids in a fed-batch manner in the 20-48^{th} hrs of fermentation is beneficial to the growth of Schizochytrium sp., wherein the dry cell weight reaches more than 150 g/L, so as to enhance the content of DHA and EPA, wherein the content of EPA in the dry cell weight can reach more than 8%. Also, with the fermentation pH adjusted from 5.5-6.0 to 5.0-6.0, and the fermentation temperature adjusted from 28° C. to 25° C. in the process of fermentation, the three culture conditions play a synergistic role with each other, allowing the content of EPA in the fermentation broth cells to reach more than 15%.

### Terminology Description

It should be noted that in this text, relational terms such as first and second are only used to distinguish one entity or operation from another entity or operation, and do not necessarily require or imply that there is any such actual relationship or order between these entities or operations. Moreover, the terms “include”, “comprise” or any other variations thereof are intended to cover non-exclusive inclusion, so that a process, method, and article including a series of elements includes not only those elements, but also other elements not explicitly listed, or elements inherent to such a process, method, and article. Without more restrictions, the elements defined by the phrase “include” or “comprise” does not exclude the existence of other elements in the process, method, and article including the elements. In addition, in this text, “greater than”, “less than”, “exceeding” and so on are understood as excluding the number itself, and “above”, “below”, “within” and so on are understood as including the number itself.

In the present application, “room temperature” and “normal temperature” refer to the ambient temperature ranging from about 10° C. to about 40° C. In some embodiments, “room temperature” or “normal temperature” refers to the temperature from about 20° C. to about 30° C.; in yet some embodiments, “room temperature” or “normal temperature” refers to the temperature from about 25° C. to about 30° C.; and in still some embodiments, “room temperature” or “normal temperature” refers to 10° C., 15° C., 20° C., 25° C., 30° C., 35° C., 40° C., etc.

In the description of this specification, descriptions referring to the terms “one embodiment”, “some embodiments”, “examples”, “specific examples” or “some examples” and so on mean that specific characteristics, structures, materials or features described in connection with this embodiment or example are included in at least one embodiment or example of the present application. In this specification, the schematic expressions of the above terms are not necessarily aimed at the same embodiment or example. Moreover, the specific characteristics, structures, materials or features described may be combined in any one or more embodiments or examples in a suitable manner. In addition, those skilled in the art can associate and combine different embodiments or examples and characteristics of different embodiments or examples described in this specification without contradicting each other.

“Inoculation amount” refers to the ratio of the volume of the transferred seed liquid to the volume of the medium after inoculation.

“g/L” is relative to the volume of a medium or the liquid in a fermentor. For example, 40-80 g/L of glucose in a medium means that each liter of the medium contains 40-80 g of glucose.

All numerical values disclosed in the present application are approximations, regardless of whether the words “about” or “approximate” are used in connection therewith. They may differ by 1%, 2%, and 5%, or sometimes even by 10% to 20%. Whenever a numerical range with a lower limit RL and an upper limit RU is disclosed, it is deemed that any numerical value falling within the range is specifically disclosed. Specifically, the following values within the range are disclosed: R=RL+k*(RU-RL), where k is a variable with a range from 1% to 100% and an increment of 1%, that is, k is 1%, 2%, 3%, 4%, 5%, . . . , 50%, 51%, 52%, 95%, 96%, 97%, 98%, 99%, or 100%. In addition, any numerical range defined by the two R numbers defined above is specifically disclosed.

“Schizochytrium sp.”, also known as Schizochytrium, belongs to a class of marine fungi in Thraustochytriidae. Schizochytrium includes but is not limited to Schizochytrium aggregatum, Schizochytrium limacinum, Schizochytrium (S31) (ATCC 20888), Schizochytrium sp. LX0809, Schizochytrium (S8) (ATCC 20889), Schizochytrium (LC-RM) (ATCC 18915), Schizochytrium (SR 21), preserved strain ATCC 28209, preserved Schizochytrium limacinum strain IFO 32693, recombinant Schizochytrium sp., Schizochytrium sp.-related strain Thraustochytrium, as well as mixtures thereof.

Adding in a fed-batch manner in the term “adding amino acids in a fed-batch manner” refers to feeding one or more amino acid complexes continuously and slowly, which can last for more than several hours according to the amount of amino acids.

### DETAILED DESCRIPTION OF THE EMBODIMENTS

The following examples are given for illustrative purposes only and are not intended to be restrictive. The following examples are illustrative of the method of the present application and ω-3 polyunsaturated fatty acids prepared by the method of the present application, but are not restrictive. Other suitable modifications and adjustments of various conditions and parameters commonly used when a seed liquid of Schizochytrium sp. is trans-inoculated to a fermentation medium for fermentation are obvious to those skilled in the art and are within the spirit and scope of the present application.

### Abbreviations

DHA: docosahexaenoic acid
DPA: docosapentaenoic acid
EPA: eicosapentaenoic acid

In the present application, the method for producing an ω-3 polyunsaturated fatty acid described in the present application is verified by taking the Schizochytrium sp. strain disclosed in China application No. 201711102734.0 as an example, but the scheme of the present application is not limited to trans-inoculating into a fermentation medium for fermentation.

The preservation information of this strain is as follows: strain name: Schizochytrium, Latin name: Schizoochytrium limacinum, strain number: HS01, preservation institution: China General Microbiological Culture Collection Center, abbreviation: CGMCC, address: No. 3, Courtyard 1, Beichen West Road, Chaoyang District, Beijing, preservation date: Mar. 10, 2017, and registration number of the preservation center: CGMCC No. 13746.

Test method:

For detection methods of the dry cell weight and total lipid, please refer to the detection method in “Study on Fermentation Technology and Metabolic Mechanism Analysis of Schizochytrium for High DHA Production”. For detection methods of the DHA content, DPA content and EPA content, please refer to and use GB 5009.168-2016, with the fatty acid area normalization method for calculation:

The dry weight and fatty acid detection methods used in Table 1 are as follows:

1. Determination method of dry cell weight:
4 mL of a fermentation broth is placed into a 10 mL centrifuge tube, and centrifuged at 4300 r/min for 3 min, and then the supernatant is removed. Additionally, 4 mL of distilled water is added, and the supernatant is removed after repeating the centrifugation operation. The precipitate is poured into a weighed plate (M), dried in an oven at 105° C., cooled and then weighed (M₁).

$$Dry weight ( g / L ) = \frac{{M}_{1} - M}{V} \times 1000$$

2. Determination of fatty acid content:

A certain mass of a fermentation broth (M₂) is weighed into a 100 mL measuring cylinder with a stopper, into which is added 10 mL of hydrochloric acid at a concentration of 8.3 mol/L, mixed evenly, placed in a water bath at 75° C. for 30 min, and taken out, into which is added 10 mL of 95% ethanol to terminate the reaction, shaken evenly, and then allowed to stand to room temperature, into which are then added 20 mL of petroleum ether and diethyl ether, respectively, and fully shaken to extract lipid. After the mixture is allowed to stand for 30 min, 5 mL of the supernatant is pipetted into a centrifugal tube with a stopper, and dried by nitrogen blowing, into which is added 2 mL of n-heptane, shaken evenly, and into which is then added 200 μL of a solution of potassium hydroxide in methanol, shaken for 1 min, kept in dark for 10 min, and centrifuged at 5000 r/min for 3 min. 1 mL of supernatant is filtered over a 0.22 m organic filter membrane, and then analyzed by gas chromatography. The fatty acid content is calculated by the normalization method.

Gas chromatography conditions: chromatographic column SP2560 (100 m×0.25 mm ID; a 0.20 μm film);
Sampling conditions: the sampling volume is 1 μL, and the split ratio is 1:10;
Carrier gas:nitrogen: 24 mL/min;
Detector temperature: 280° C.; and
Column temperature conditions: the initial temperature is kept at 100° C. for 13 min, and then raised to 180° C. at 10° C./min and kept for 6 min; then raised to 200° C. at 1° C./min and kept for 20 min; and then raised to 230° C. at 4° C./min and kept for 10.5 min.

Calculation formula of EPA content (Y₁):

$${Y}_{1} = \frac{{F}_{i}}{{F}_{total}}$$

In the formula, Y₁ is the content of a single fatty acid (%);
Fᵢ is a peak area of the fatty acid methyl ester; and
Fₜₒₜₐₗ is the total peak area of the fatty acid methyl ester.

### 3. Determination Method of Residual Sugar (Glucose)

### 1. Reagents

Potassium sodium tartrate, dinitrosalicylic acid, sodium hydroxide, phenol, sodium sulfite, and glucose standard

### 2. Experimental Equipment

A 10 mL centrifuge tube, a volumetric flask, and a spectrophotometer

### 3. Glucose is Detected by 3,5-Dinitrosalicylic Acid (DNS) Colorimetry

### 3.1 Formulation of DNS Solution

185 g of potassium sodium tartrate is accurately weighed, into which is added water to a constant volume of 500 mL and heated for later use. 6.3 g of dinitrosalicylic acid is then weighed and 262 mL of 2 mol/L NaOH solution is measured into the hot solution of potassium sodium tartrate, and fully stirred and mixed evenly; next, 5 g of phenol and 5 g of sodium sulfite are additionally added into the mixture, fully stirred and dissolved, and the solution is placed into a brown bottle for later use.

### 3.2 Drawing of Glucose Standard Curve

0.01 g, 0.02 g, 0.03 g, 0.04 g and 0.05 g of the glucose standard are weighed, into which is added water to a constant volume of 50 mL. 1 mL of the glucose diluent and 2 ml of the DNS solution are added into a colorimetric tube with a stopper, heated with boiling water for 2 min, and cooled, into which is then added 9 mL of distilled water, and shaken well. The absorbance is determined at a wavelength of 540 nm. Taking the sample without the glucose standard as the control group, the standard curve is drawn with the glucose concentration as the abscissa and the absorbance as the ordinate.

In the present application, there is provided a method for producing an ω-3 polyunsaturated fatty acid, including:
(1) culturing Schizochytrium sp. to obtain a seed liquid of Schizochytrium sp.; and
(2) trans-inoculating the seed liquid of Schizochytrium sp. obtained in step (1) into a fermentation medium in an inoculation amount of 30-40% for fermentation at conditions as below: a fermentation temperature of 25-28° C., and a fermentation pH of 5.0-6.0, the pH and temperature of the fermentation are regulated in stages, a concentration of a residual sugar in the fermentation medium during the fermentation is maintained at 20-40 g/L by batch feeding, dissolved oxygen in the fermentation process is controlled at more than 50%, and a total fermentation time is 144-168 hrs, to obtain the ω-3 polyunsaturated fatty acid, wherein 3-10 g/L of amino acids are added in a fed-batch manner at the 20-48 hr stage after the start of the fermentation.

### Example 1: Preparation of Seed Liquid of Schizochytrium sp.

The culture process of the seed liquid of Schizochytrium sp. is as follows:

### 1. Shake-Flask Seed Culture

10 ml of Schizochytrium strains in a glycerol tube preserved in an ultra-low temperature refrigerator was inoculated into a 250 ml shake flask containing 50 ml of medium, and cultured in a shaker at 25° C. at a rotation speed of 220 rpm for 24 hrs; and then inoculated into a 1000 ml shake flask containing 400 ml of the medium in an inoculation amount of 10%, and cultured in a shaker at 25° C. at a rotation speed of 220 rpm for 24 hrs to obtain a shake-flask seed liquid.

Shake-flask seed medium: 40-80 g/L of glucose, 10-15 g/L of yeast extract powder, 10-15 g/L of anhydrous sodium sulfate, 0.1-0.2 g/L of anhydrous calcium chloride, 0.3-0.6 g/L of potassium chloride, 0.5-1.5 g/L of potassium dihydrogen phosphate, 2.0-4.0 g/L of ammonium sulfate, 1.0-4.0 g/L of zinc sulfate, 2.0-6.0 g/L of magnesium sulfate, and 0.5-1.5 g/L of potassium sulfate; and
culture conditions of shake-flask seed culture: shake-flask culturing at 28-30° C. and 180-220 rpm for 24-30 hrs.

### 2. Primary Seed Culture

The shake-flask seed liquid was inoculated into a primary seed medium in an inoculation amount of 2-4% for culture, and cultured until the residual sugar in the primary seed medium was less than 20 g/L to obtain a primary seed liquid;
primary seed medium: 40-80 g/L of glucose, 5-15 g/L of yeast extract powder, 10-15 g/L of anhydrous sodium sulfate, 0.1-0.2 g/L of anhydrous calcium chloride, 0.3-0.6 g/L of potassium chloride, 0.5-1.5 g/L of potassium dihydrogen phosphate, 2.0-4.0 g/L of ammonium sulfate, 1.0-4.0 g/L of zinc sulfate, 2.0-6.0 g/L of magnesium sulfate, and 0.5-1.5 g/L of potassium sulfate; and
conditions of primary seed culture: 28-30° C., adjusting the pH of the fermentation to 5.0-6.0 with a base and an acid, and culturing for 24-30 hrs, for example, using ammonia water with a certain concentration to adjust pH.

### 3. Secondary Seed Culture

The primary seed liquid was inoculated into a secondary seed medium in an inoculation amount of 10-20% for culture, and cultured until the residual sugar in the secondary seed medium was less than 20 g/L to obtain a seed liquid of Schizochytrium sp.;
secondary seed medium: 40-60 g/L of glucose, 5-15 g/L of yeast extract powder, 5-10 g/L of yeast powder, 10-20 g/L of anhydrous sodium sulfate, 0.1-0.2 g/L of anhydrous calcium chloride, 0.3-0.6 g/L of potassium chloride, 0.5-1.5 g/L of potassium dihydrogen phosphate, 2.0-4.0 g/L of ammonium sulfate, 1.0-4.0 g/L of zinc sulfate, 2.0-6.0 g/L of magnesium sulfate, and 0.5-1.5 g/L of potassium sulfate, dissolving the above components with purified water; and
culture conditions of secondary seed culture: 28-30° C., adjusting the pH of the fermentation to 5.0-6.0 with a base and an acid, and culturing for 24-30 hrs.

The seed liquid of Schizochytrium sp. obtained from the above secondary seed culture was used to carry out the experiments in following examples:

### Example 2

The seed liquid of Schizochytrium sp. obtained in Example 1 was trans-inoculated into a fermentation medium in an inoculation amount of 37.5% for fermentation at conditions as below: a fermentation temperature of 28° C. and a fermentation pH of 5.5-6.0, the concentration of the residual sugar was maintained at 20-40 g/L by batch feeding, dissolved oxygen in the fermentation process was controlled at more than 50%, and the total fermentation time was 144 hrs, to obtain a fermentation broth, wherein 5 g/L of complex amino acids (lysine:glycine=1:1) was added in a fed-batch manner at the 20-48 hr stage of the fermentation.

The fermentation medium was: 50 g/L of glucose, 12 g/L of yeast extract powder, 3 g/L of yeast powder, 5 g/L of corn syrup dry powder, 15 g/L of anhydrous sodium sulfate, 0.17 g/L of anhydrous calcium chloride, 0.5 g/L of potassium chloride, 0.65 g/L of magnesium sulfate, 1 g/L of potassium dihydrogen phosphate, 1 g/L of ammonium sulfate, 20 mg/L of Vitamin B12, 10 mg/L of Vitamin B1, and 10 mg/L of biotin.

After fermentation for 144 hrs, the dry cell weight of the fermentation broth was determined, the content ratio of the crude lipid was also determined, and the content of the related ω-3 polyunsaturated fatty acid in the crude lipid was determined.

### Example 3

The seed liquid of Schizochytrium sp. obtained in Example 1 was trans-inoculated into a fermentor at 30%. Compared with Example 2, the differences were as below:

The total fermentation time was 144 hrs, and 3 g/L of complex amino acids (glutamic acid:lysine:tyrosine=1:1:1) were added in a fed-batch manner at the 20-48 hr stage of the fermentation.

The fermentation medium was: 40 g/L of glucose, 15 g/L of yeast extract powder, 2 g/L of yeast powder, 5 g/L of corn syrup dry powder, 15 g/L of anhydrous sodium sulfate, 0.1 g/L of anhydrous calcium chloride, 0.3 g/L of potassium chloride, 0.65 g/L of magnesium sulfate, 1 g/L of potassium dihydrogen phosphate, 0.5 g/L of ammonium sulfate, 40 mg/L of Vitamin B12, 10 mg/L of Vitamin B1, and 10 mg/L of biotin.

### Example 4

The seed liquid of Schizochytrium sp. obtained in Example 1 was trans-inoculated into a fermentor at 35%. Compared with Example 2, the differences were as below:

The total fermentation time was 144 hrs, and 10 g/L of complex amino acids (the mass ratio of lysine:threonine:arginine is 1:0.5:0.5) were added in a fed-batch manner at the 20-48 hr stage of the fermentation

The fermentation medium was: 60 g/L of glucose, 5 g/L of yeast extract powder, 5 g/L of yeast powder, 2 g/L of corn syrup dry powder, 10 g/L of anhydrous sodium sulfate, 0.2 g/L of anhydrous calcium chloride, 0.3 g/L of potassium chloride, 0.4 g/L of magnesium sulfate, 0.5 g/L of potassium dihydrogen phosphate, 0.5 g/L of ammonium sulfate, 15 mg/L of Vitamin B12, 5 mg/L of Vitamin B1, and 5 mg/L of biotin.

### Example 5

The seed liquid of Schizochytrium sp. obtained in Example 1 was trans-inoculated into a fermentor at 40%. Compared with Example 2, the differences were as below:

The total fermentation time was 168 hrs, and 10 g/L of complex amino acids (the mass ratio of lysine:glycine:phenylalanine is 1:1:1) were added in a fed-batch manner at the 20-48 hr stage of the fermentation.

The fermentation medium was: 50 g/L of glucose, 15 g/L of yeast extract powder, 5 g/L of yeast powder, 10 g/L of corn syrup dry powder, 30 g/L of anhydrous sodium sulfate, 0.2 g/L of anhydrous calcium chloride, 0.6 g/L of potassium chloride, 0.8 g/L of magnesium sulfate, 1.5 g/L of potassium dihydrogen phosphate, 1.5 g/L of ammonium sulfate, 40 mg/L of Vitamin B12, 15 mg/L of Vitamin B1, and 15 mg/L of biotin.

### Comparative Example 1

The seed liquid of Schizochytrium sp. obtained in Example 1 was trans-inoculated into a fermentor at 37.5%. Compared with Example 2, the differences were as below:

The total fermentation time was 144 hrs, and no amino acid was added in a fed-batch manner during the whole fermentation.

The remaining steps were the same as in Example 2.

### Comparative Example 2

The seed liquid of Schizochytrium sp. obtained in Example 1 was trans-inoculated into a fermentor at 37.5%. Compared with Example 2, the differences were as below:

The total fermentation time was 144 hrs, and 5 g/L of complex amino acids (lysine:glycine=1:1) were added in a fed-batch manner at the 20-144 hr stage of the fermentation.

The remaining steps were the same as in Example 2.

The detection results of the fermentation broths of Examples 2-5 and Comparative Examples 1-2 are shown in Table 1 below:

TABLE 1
Fermentation results of Examples
2-5 and Comparative Examples 1-2.

| Experimental | Dry cell | Palmitic | DHA | EPA | DPA |
|---|---|---|---|---|---|
| groups | weight (g/L) | acid (%) | (%) | (%) | (%) |
| Example 2 | 165 | 13.33 | 40.12 | 9.52 | 3.57 |
| Example 3 | 152 | 13.26 | 37.76 | 8.04 | 3.23 |
| Example 4 | 157 | 13.64 | 38.57 | 9.13 | 3.45 |
| Example 5 | 162 | 12.78 | 38.12 | 8.67 | 3.48 |
| Comparative | 123 | 14.38 | 35.27 | 3.34 | 2.56 |
| Example 1 | | | | | |
| Comparative | 133 | 18.67 | 37.59 | 4.35 | 3.05 |
| Example 2 | | | | | |

Among them, palmitic acid (0%), DHA (0), EPA (0%) and DPA (0%) represent the contents of corresponding unsaturated fatty acids in the cell lipid, respectively.

By comparing Example 2-5 with Comparative Example 1, it can be seen that the dry cell weight of the fermentation broth after fermentation can be effectively enhanced by adding amino acids in a fed-batch manner in the fermentation step in the present application, which is also helpful to enhance the contents of DHA and EPA produced from Schizochytrium sp.

By comparing Example 2-5 with Comparative Example 2, it can be seen that the timing of adding amino acids in a fed-batch manner in the present application is determined as the 20-48 hr stage of the fermentation. Compared with the long-term addition in a fed-batch manner in Comparative Example 2, the method of the present application only add amino acids in a fed-batch manner in a short time, which reduces the fermentation cost, and the final fermentation effects (the dry cell weight and the contents of unsaturated fatty acids) are also better than those in Comparative Example 2.

### Example 6

The seed liquid of Schizochytrium sp. obtained in Example 1 was trans-inoculated into a fermentor, and the temperature of the fermentation culture conditions was adjusted relative to Example 2.

The culture conditions of the fermentor were as below: the temperature was controlled at 28° C. for the first 48 hrs of fermentation, and after 48 hrs, the temperature was reduced to 25° C.

### Example 7

The seed liquid of Schizochytrium sp. obtained in Example 1 was trans-inoculated into a fermentor, and the pH of the fermentation culture environment was adjusted relative to Example 2.

The fermentation culture conditions were as below: the pH was controlled at 5.5-6.0 for the first 48 hrs of fermentation, and after 48 hrs, the pH was adjusted to 5.0-6.0; the concentration of the residual sugar was maintained at 20-40 g/L by batch feeding, and dissolved oxygen in the fermentation process was controlled at more than 50%.

The pH was adjusted with an acid and a base in the fermentation process, and the base was a complex of one or more of ammonia water, sodium hydroxide, potassium hydroxide, sodium carbonate and calcium carbonate; and the acid was a complex of one or more of citric acid, malic acid, acetic acid, propionic acid, succinic acid, oxalic acid, phosphoric acid, sulfuric acid, hydrochloric acid, and phytic acid.

### Example 8

The seed liquid of Schizochytrium sp. obtained in Example 1 was trans-inoculated into a fermentation medium, and the temperature and pH of the fermentation culture conditions were adjusted relative to Example 2.

The fermentation culture conditions were as below: the temperature was controlled at 28° C., and the pH was controlled at 5.5-6.0 for the first 48 hrs of fermentation, and after 48 hrs, the temperature was reduced to 25° C., and the pH was adjusted to 5.0-6.0.

### Comparative Example 3

The seed liquid of Schizochytrium sp. obtained in Example 1 was trans-inoculated into a fermentation medium. Compared with Example 2, the differences were as below: the temperature and pH of the fermentation culture conditions were adjusted in the fermentation process, and no amino acid was added in a fed-batch manner during the whole fermentation.

The fermentation culture conditions were as below: the temperature was controlled at 28° C., and the pH was controlled at 5.5-6.0 for the first 48 hrs of fermentation, and after 48 hrs, the temperature was reduced to 25° C., and the pH was adjusted to 5.0-6.0.

The detection results of the fermentation broths of Examples 6-8 and Comparative Examples 3 are shown in Table 2 below:

TABLE 2
Fermentation results of Examples
6-8 and Comparative Examples 3.

| Experimental | Dry cell | Palmitic | DHA | EPA | DPA |
|---|---|---|---|---|---|
| group | weight (g/L) | acid (%) | (%) | (%) | (%) |
| Example 6 | 168 | 12.75 | 38.76 | 9.86 | 3.75 |
| Example 7 | 185 | 12.84 | 41.24 | 10.31 | 4.54 |
| Example 8 | 209 | 12.66 | 44.53 | 15.15 | 5.78 |
| Comparative | 143 | 13.16 | 37.28 | 5.23 | 3.42 |
| Example 3 | | | | | |

Among them, palmitic acid (%), DHA (%), EPA (%) and DPA (%) represent the contents of corresponding unsaturated fatty acids in the dry cell weight, respectively.

By comparing Example 6-8 with Comparative Example 3, it can be seen that, in the present application, by adding complex amino acids in a fed-batch manner in the fermentation step, while adjusting the pH and temperature in the fermentation process, the three culture conditions can play a synergistic role with each other, thus enhancing the dry cell weight (up to 209 g/L) and the EPA content (the content can be up to 15.15%).

Although the various embodiments have been described above, those skilled in the art can make other changes and modifications to these embodiments once they know the basic creative concepts. Therefore, those described above are only embodiments of the present application, which do not limit the patent protection scope of the present application. All equivalent structures or equivalent process changes made by using the contents of the specification and drawings of the present application, or directly or indirectly applied to other related technical fields, are all equally included in the patent protection scope of the present application.

What is claimed is:

1. A method for producing an ω-3 polyunsaturated fatty acid, comprising: culturing Schizochytrium sp. to obtain a seed liquid of Schizochytrium sp.; and trans-inoculating the obtained seed liquid of Schizochytrium sp. into a fermentation medium for fermentation, wherein pH and temperature of the fermentation are regulated in stages, a concentration of a residual sugar in the fermentation medium during the fermentation is maintained at 20-40 g/L by batch feeding, and dissolved oxygen in a fermentation process is controlled at more than 20%.

2. The method according to claim 1, comprising adding amino acids in a fed-batch manner at a stage of about 20-48 hr after the start of the fermentation.

3. The method for producing an ω-3 polyunsaturated fatty acid according to claim 1, comprising:
(1) culturing Schizochytrium sp. to obtain a seed liquid of Schizochytrium sp.; and
(2) trans-inoculating the seed liquid of Schizochytrium sp. obtained in step (1) into a fermentation medium in an inoculation amount of 30-40% for fermentation at conditions as below: a fermentation temperature of 25-28° C., and a fermentation pH of 5.0-6.0, the pH and temperature of the fermentation are regulated in stages, a concentration of a residual sugar in the fermentation medium during the fermentation is maintained at 20-40 g/L by batch feeding, dissolved oxygen in the fermentation process is controlled at more than 50%, and a total fermentation time is 144-168 hrs, to obtain the ω-3 polyunsaturated fatty acid, wherein 3-10 g/L of amino acids are added in a fed-batch manner at a stage of 20-48 hr after the start of the fermentation.

4. The method according to claim 1, wherein the amino acids are at least two of glutamic acid, lysine, tyrosine, threonine, arginine, glycine, and phenylalanine, and the batch feeding is to add glucose as a supplement.

5. The method according to claim 1, wherein the process of regulating the pH of the fermentation in stages comprises controlling the pH to be 5.5-6.0 during a 0-48 hr stage of the fermentation process; and controlling the pH to be 5.0-6.0 after 48 hrs of fermentation.

6. The method according to claim 4, wherein the pH is adjusted with an acid and/or a base in the fermentation process, the base is a complex of one or more of ammonia water, sodium hydroxide, potassium hydroxide, sodium carbonate and calcium carbonate; and the acid is a complex of one or more of citric acid, malic acid, acetic acid, propionic acid, succinic acid, oxalic acid, phosphoric acid, sulfuric acid, hydrochloric acid and phytic acid.

7. The method according to claim 1, wherein the process of regulating the temperature of the fermentation in stages comprises controlling the fermentation temperature to be 28° C. during a 0-48 hr stage of the fermentation process, and controlling the fermentation temperature to be 25° C. after 48 hrs of fermentation.

8. The method according to claim 1, wherein the fermentation medium comprises: 40-60 g/L of initial glucose, 5-15 g/L of yeast extract powder, 2-5 g/L of yeast powder, 2-10 g/L of corn syrup dry powder, 10-30 g/L of anhydrous sodium sulfate, 0.1-0.2 g/L of anhydrous calcium chloride, 0.3-0.6 g/L of potassium chloride, 0.4-0.8 g/L of magnesium sulfate, 0.5-1.5 g/L of potassium dihydrogen phosphate, 0.5-1.5 g/L of ammonium sulfate, 15-40 mg/L of Vitamin B12, 5-15 mg/L of Vitamin B1, and 5-15 mg/L of biotin.

9. The method according to claim 1, wherein in step (1), Schizochytrium sp. is cultured by a shake-flask culture, a primary seed culture and a secondary seed culture to obtain the seed liquid of Schizochytrium sp.,
wherein:
the shake-flask culture comprises: inoculating Schizochytrium sp. into a shake-flask seed medium for culture to obtain a shake-flask seed liquid, wherein culture conditions of the shake-flask culture are as follows: culturing at a temperature of 28-30° C. and in a shake flask at 180-220 rpm for 24-30 hrs;
the primary seed culture comprises: inoculating the shake-flask seed liquid into a primary seed medium in an inoculation amount of 24% to obtain a primary seed liquid; wherein culture conditions of the primary seed culture are as follows: culturing at a temperature of 28-30° C. and adjusting the pH of the fermentation to 5.0-6.0 with a base and/or an acid; and
the secondary seed culture comprises: after culturing in the primary seed culture step to a residual sugar concentration in the primary seed medium of less than 20 g/L, inoculating the primary seed liquid into a secondary seed medium in an inoculation amount of 10-20% for culture to obtain the seed liquid of Schizochytrium sp.; wherein culture conditions of the secondary seed culture are as follows: culturing at a temperature of 28-30° C., adjusting the pH of the fermentation to 5.0-6.0 with a base and/or an acid, and culturing the secondary seed until the residual sugar in the secondary seed medium is less than 20 g/L.

10. The method according to claim 9, wherein
the shake-flask seed medium comprises: 40-80 g/L of glucose, 10-15 g/L of yeast extract powder, 10-15 g/L of anhydrous sodium sulfate, 0.1-0.2 g/L of anhydrous calcium chloride, 0.3-0.6 g/L of potassium chloride, 0.5-1.5 g/L of potassium dihydrogen phosphate, 2.0-4.0 g/L of ammonium sulfate, 1.0-4.0 g/L of zinc sulfate, 2.0-6.0 g/L of magnesium sulfate, and 0.5-1.5 g/L of potassium sulfate;
the primary seed medium comprises: 40-80 g/L of glucose, 5-15 g/L of yeast extract powder, 10-15 g/L of anhydrous sodium sulfate, 0.1-0.2 g/L of anhydrous calcium chloride, 0.3-0.6 g/L of potassium chloride, 0.5-1.5 g/L of potassium dihydrogen phosphate, 2.0-4.0 g/L of ammonium sulfate, 1.0-4.0 g/L of zinc sulfate, 2.0-6.0 g/L of magnesium sulfate, and 0.5-1.5 g/L of potassium sulfate; and
the secondary seed medium comprises: 40-60 g/L of glucose, 5-15 g/L of yeast extract powder, 5-10 g/L of yeast powder, 10-20 g/L of anhydrous sodium sulfate, 0.1-0.2 g/L of anhydrous calcium chloride, 0.3-0.6 g/L of potassium chloride, 0.5-1.5 g/L of potassium dihydrogen phosphate, 2.0-4.0 g/L of ammonium sulfate, 1.0-4.0 g/L of zinc sulfate, 2.0-6.0 g/L of magnesium sulfate, and 0.5-1.5 g/L of potassium sulfate.

11. The method according to claim 1, wherein the ω-3 polyunsaturated fatty acid comprises at least one of eicosapentaenoic acid, docosapentaenoic acid or docosahexaenoic acid.

12. The method according to claim 11, wherein the eicosapentaenoic acid is at least more than 8.0% by weight in the unsaturated fatty acid.
